(19) United States
(12) Patent Application Publication
(10) Pub. No.: US 2024/0221871 A1
(43) Pub. Date: Jul. 4, 2024

(54) METHOD AND DEVICE FOR MEASURING CONCENTRATION OF VOLATILE ORGANIC COMPOUNDS (VOCS), AND READABLE STORAGE MEDIUM

(71) Applicant: SHENZHEN PUREMATE TECHNOLOGY CO., LTD., Shenzhen (CN)

(72) Inventors: Dequan LIU, Shenzhen (CN); Yiqing YAN, Shenzhen (CN); Yuyang LIU, Shenzhen (CN); Fangsheng YAN, Shenzhen (CN)

(73) Assignee: SHENZHEN PUREMATE TECHNOLOGY CO., LTD., Shenzhen (CN)

(21) Appl. No.: 18/604,190

(22) Filed: Mar. 13, 2024

Related U.S. Application Data
(63) Continuation of application No. PCT/CN2022/092718, filed on May 13, 2022.

(30) Foreign Application Priority Data
Sep. 30, 2021 (CN) 202111169323.X

(51) Int. Cl. G16C 20/20; G01N 21/3504; G01N 30/02; G01N 30/06; G01N 30/88

(52) U.S. Cl. CPC G16C 20/20; G01N 21/3504; G01N 30/02; G01N 2030/025; G01N 2030/067; G01N 2030/884

(57) ABSTRACT

Disclosed are a method and a device for measuring a concentration of volatile organic compounds (VOCs), and a readable storage medium. The method includes: starting a sampling device to inject the VOCs of a sample gas to be measured into a gas-phase electrochemical reactor for performing gas-phase electrochemical oxidation, and converting the VOCs into carbon dioxide (CO₂); and measuring a target conversion concentration of CO₂ to obtain a carbon concentration of the VOCs in the sample gas to be measured.

### CROSS-REFERENCE TO RELATED APPLICATIONS

This application is a continuation of International Application No. PCT/CN2022/092718, filed on May 13, 2022, which claims priority to Chinese Patent Application No. 202111169323.X, filed on Sep. 30, 2021. All of the aforementioned applications are incorporated herein by reference in their entireties.

### TECHNICAL FIELD

The present application relates to the technical field of organic compound measurement, and in particular to a method and a device for measuring a concentration of volatile organic compounds (VOCs), and a readable storage medium.

### BACKGROUND

Currently, the volatile organic compounds (VOCs) on the market are mainly measured by the gas chromatography-flame ionization detector (GC-FID).

However, high-purity hydrogen needs to be used during the measurement process, and high-purity hydrogen is an extremely flammable gas, so there is a high safety risk during the process of measuring the concentration of VOCs.

### SUMMARY

The main purpose of the present application is to provide a method and a device for measuring a concentration of volatile organic compounds (VOCs), and a readable storage medium, aiming to solve the technical problem of how to reduce safety risks during the process of measuring the concentration of VOCs.

In order to achieve the above objectives, the present application provides a method for measuring a concentration of VOCs, which includes:
starting a sampling device to inject the VOCs of a sample gas to be measured into a gas-phase electrochemical reactor for performing gas-phase electrochemical oxidation, and converting the VOCs into carbon dioxide (CO₂); and
measuring a target conversion concentration of CO₂ to obtain a carbon concentration of the VOCs in the sample gas to be measured.

In an embodiment, before the starting the sampling device to inject the VOCs of the sample gas to be measured into the gas-phase electrochemical reactor for performing gas-phase electrochemical oxidation, and converting the VOCs into CO₂, the method further includes:
measuring an original concentration of CO₂ in the sample gas to be measured based on an infrared optical platform; and
the measuring the target conversion concentration of CO₂ includes measuring a target conversion concentration of CO₂ based on the original concentration.

In an embodiment, the measuring the target conversion concentration of CO₂ based on the original concentration includes:
measuring a total concentration of CO₂ after conversion based on the infrared optical platform; and
calculating a difference between the total concentration after conversion and the original concentration to obtain the target conversion concentration.

In an embodiment, the measuring the original concentration of CO₂ in the sample gas to be measured based on the infrared optical platform includes:
controlling an infrared light source to emit an infrared light with a preset wavelength to a gas chamber where the sample gas to be measured is located; and
obtaining an intensity after an infrared detector detects the infrared light, and calculating the original concentration of CO₂ in the sample gas to be measured based on the intensity.

In an embodiment, before the starting the sampling device to inject the VOCs of the sample gas to be measured into the gas-phase electrochemical reactor for performing gas-phase electrochemical oxidation, and converting the VOCs into CO₂, the method further includes:
calculating an influence value corresponding CO₂ in air; and
in response to that the influence value meets a preset requirement, performing the starting the sampling device to inject the VOCs of the sample gas to be measured into the gas-phase electrochemical reactor for performing gas-phase electrochemical oxidation, and converting the VOCs into CO₂.

In an embodiment, the influence value includes a zero point influence value and a range point influence value, and the calculating the influence value corresponding CO₂ in air includes:
calculating a zero point influence value corresponding to a zero gas with a first preset concentration and a range point influence value corresponding to a range standard gas with a second preset concentration respectively.

In an embodiment, the VOCs are multi-component organic compounds, and the starting the sampling device to inject the VOCs of the sample gas to be measured into the gas-phase electrochemical reactor for performing gas-phase electrochemical oxidation, and converting the VOCs into CO₂ includes:
starting the sampling device to separate the multi-component organic compounds into a plurality of single-component organic compounds based on a chromatographic column, and injecting each of the plurality of single-component organic compounds into the gas-phase electrochemical reactor respectively to perform gas-phase electrochemical oxidation and convert the single-component organic compound into CO₂.

In an embodiment, the method further includes measuring a single-component conversion concentration of CO₂ corresponding to each of the plurality of single-component organic compounds respectively, to obtain a gas chromatogram.

In order to achieve the above objectives, the present application further provides a device for measuring a concentration of VOCs, including a memory, a processor, and a program for measuring the concentration of the VOCs stored on the memory and executable on the processor. When the program is executed by the processor, the method as mentioned above is implemented.

In order to achieve the above objectives, the present application further provides a non-transitory readable storage medium. A program for measuring the concentration of the VOCs is stored on the non-transitory readable storage medium, and when the program is executed by a processor, the method as mentioned above is implemented.

The existing technology uses high-purity hydrogen to measure the concentration of VOCs, resulting in higher safety risks in the process of measuring the concentration of VOCs. Compared with that, in the present application, the sampling device is started to inject the VOCs in the sample gas to be measured to the gas-phase electrochemical reactor for performing gas-phase electrochemical oxidation and converting it into CO₂. The target conversion concentration of CO₂ is measured to obtain the carbon concentration of the VOCs in the sample gas to be measured. In the present application, the VOCs can be converted into CO₂ through a gas-phase electrochemical reactor. The gas-phase electrochemical reactor does not need to use the high-purity hydrogen when being used, so it does not involve the issue of safety management of flammable gases, thereby reducing the security risks in the process of measuring the concentration of VOCs, and the method is easy to implement.

### BRIEF DESCRIPTION OF THE DRAWINGS

To more clearly illustrate technical solutions in the embodiments of the present application or the related art, the following will briefly introduce the drawings that need to be used in the description of the embodiments or the related art.

FIG. 1 is a schematic flowchart of the method for measuring the concentration of the VOCs according to an embodiment of the present application.

FIG. 2 is a schematic diagram of the flow direction of the sample gas to be measured in the measurement system according to an embodiment of the present application.

FIG. 3 is a schematic diagram of the measurement principle of measuring the concentration of CO₂ according to an embodiment of the present application.

FIG. 4 is a schematic diagram of the functional module in the device for measuring the concentration for the VOCs according to an embodiment of the present application.

FIG. 5 is a schematic structural diagram of the hardware operating environment according to an embodiment of the present application.

The realization of the objective, functional characteristics, and advantages of the present application are further described with reference to the accompanying drawings.

### DETAILED DESCRIPTION OF THE EMBODIMENTS

The technical solutions of the embodiments of the present application will be described in detail below. It is obvious that the embodiments described are only some rather than all of the embodiments of the present application. All other embodiments obtained by those skilled in the art based on the embodiments of the present application without creative efforts shall fall within the claimed scope of the present application.

In addition, the technical solutions of the various embodiments can be combined with each other, but the combinations must be based on the realization of those skilled in the art. When the combination of technical solutions is contradictory or cannot be achieved, it should be considered that such a combination of technical solutions does not exist, nor does it fall within the scope of the present application.

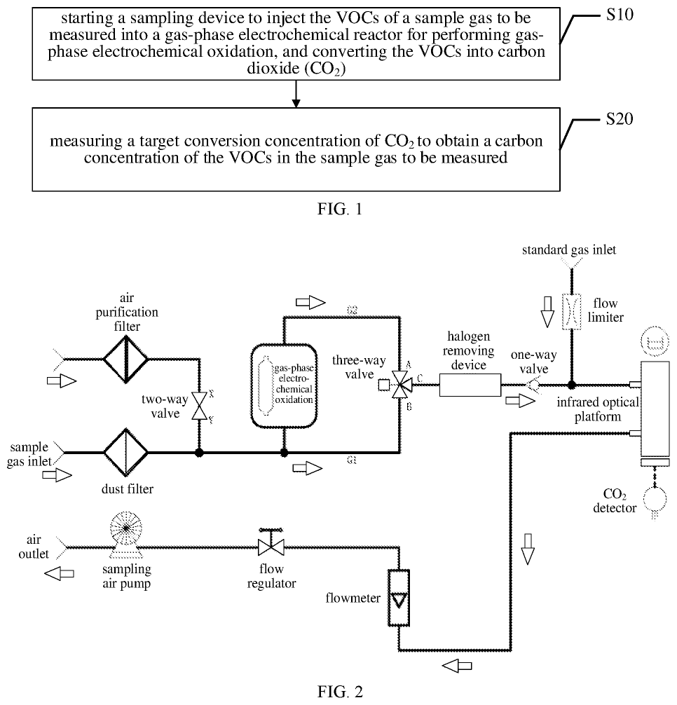

The present application provides a method for measuring the concentration of the volatile organic compounds (VOCs). As shown in FIG. 1, FIG. 1 is a schematic flowchart of the method for measuring the concentration of the VOCs according to an embodiment of the present application.

The present application provides embodiments of the method for measuring the concentration of the VOCs. It should be noted that although the logical sequence is shown in the flowchart, in some cases, the steps shown can be performed in a sequence different from that shown here. The method for measuring the concentration of the VOCs can be applied to the personal computer, the terminal, and the like. For convenience of description, the executive body is omitted below to describe each step of the method for measuring the concentration of the VOCs. The method for measuring the concentration of the VOCs includes:
step S10, starting a sampling device to inject the VOCs of a sample gas to be measured into a gas-phase electrochemical reactor for performing gas-phase electrochemical oxidation, and converting the VOCs into carbon dioxide (CO₂).

In this embodiment, as shown in FIG. 2, FIG. 2 is a schematic diagram of the flow direction of the sample gas to be measured in the measurement system (a system adopting the extraction measurement mode) according to an embodiment of the present application. The measurement system consists of a sampling device (the sample collection and transmission device), a preprocessing equipment, a gas-phase electrochemical oxidation and non-dispersive infrared measurement unit, an exhaust gas parameter monitoring unit, a data collection and transmission equipment, an auxiliary equipment, and the like. The sample collection and transmission device mainly includes a sampling probe, a sample transmission pipeline, a flow control equipment, and a sampling air pump. It can be understood that the instrument adopting an extraction measurement method is generally equipped with the sample collection and transmission device. The preprocessing equipment mainly includes a particulate filter, namely the air purification filter and the dust filter shown in FIG. 2. The gas-phase electrochemical oxidation and non-dispersive infrared measurement unit is a unit that adopts the electrochemical technology to convert VOCs into CO₂ and then performs measurement, which includes the gas-phase electrochemical oxidation and infrared optical platform shown in FIG. 2. The exhaust gas parameter monitoring unit is configured to measure parameter information such as the exhaust gas temperature, the pressure, the flow rate (or the flow) and the humidity. The data collection and transmission equipment is configured to collect, process and store the measurement data, and can transmit measurement data and equipment working state information according to the instructions of the central computer (such as the personal computer, the terminal, and the like). The auxiliary equipment mainly includes a blowback purification device and a control equipment thereof, and a zero air preprocessing equipment, and the like.

It should be noted that the overall measurement system can not only measure the concentration of VOCs in the exhaust gas, the exhaust gas parameter (the temperature, the pressure, the flow rate or the flow, the humidity, and the like), but also can calculate the emission rate and the emission amount of pollutants in the exhaust gas and can display various parameters and charts corresponding to the exhaust gas. The on-site law enforcement and online data can be transmitted to the management department through data, graphics and text.

It should be noted that the method for measuring the concentration of the VOCs in the present application is a control method of the measurement system.

It should be noted that in the process that the electrochemical technology is adopted to convert VOCs other than methane into CO₂, the VOCs other than the methane need to be completely converted into CO₂, but this conversion process cannot be fully implemented by the current VOC catalytic oxidation technology at a normal temperature and a pressure, and the CO₂ conversion rate is low. The gas-phase electrochemical reactor used in the present application can completely convert VOCs other than methane into CO₂ at the normal temperature and pressure. The gas-phase electrochemical reactor includes a power supply, an anode, a cathode, and a proton exchange membrane. The proton exchange membrane is provided between the anode and the cathode, and the anode, proton exchange membrane and cathode are clamped.

It should be noted that in addition to safety issues, the flame ionization detector (FID) further needs to be equipped with devices such as the carrier gas device, the fuel gas device, and the filtration device during the measurement process, which makes the measurement process inconvenient. In addition, the device further requires professional technical personnel to operate and maintain, and has higher requirements for the operating environment of the equipment. It can be understood that the FID cannot perform online measurement and on-site law enforcement. Since the measurement system of the present application does not use the high-purity hydrogen, the structure of the measurement system is relatively simple and easy to operate and maintain, and has low requirements for the professional personnel to operate and maintain. In addition, the measurement process of the present application does not consume chemicals, hydrogen, and does not require auxiliary facilities such as the carrier gas device, the measurement process have relatively low requirements on the space and environment of the testing site if the measurement system of the present application is used as the online monitoring equipment, and has a low operating cost, which can be operated in situations with harsh working conditions.

It should be noted that the gas-phase electrochemical reactor can oxidize different types of organic compound and convert it into CO₂. The chemical reaction formula is:

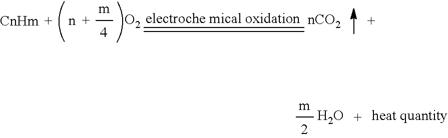

It can be understood that in this redox process, VOCs is oxidized into CO₂ and H2O and reaction heat is released.

Step S20, measuring a target conversion concentration of CO₂ to obtain a carbon concentration of the VOCs in the sample gas to be measured.

In this embodiment, it can be seen from the above chemical reaction formula that according to the principle of conservation of matter, the carbon concentration corresponding to CO₂, which is one of the products of oxidation, directly reflects the carbon concentration of the organic compound participating in the reaction. Therefore, by measuring the concentration increment of CO₂ before and after the oxidation reaction respectively, the concentration of VOCs can be measured.

Further, before the starting the sampling device to inject the VOCs of the sample gas to be measured into the gas-phase electrochemical reactor for performing gas-phase electrochemical oxidation, and converting the VOCs into CO₂, the method for measuring the concentration further includes:
step a, measuring an original concentration of CO₂ in the sample gas to be measured based on an infrared optical platform.

In this embodiment, by measuring the original concentration of CO₂ in the sample gas to be measured, the influence of the original CO₂ in the sample gas to be measured on the measurement results can be eliminated.

It should be noted that, as shown in FIG. 2, the process of measuring the original concentration of CO₂ in the sample gas to be measured is implemented through the following process. First, the terminal X of the two-way solenoid valve is controlled to disconnect from the terminal Y of the two-way solenoid valve, the C terminal of the three-way valve is controlled to connect with the terminal B of the three-way valve, the terminal C of the three-way valve is controlled to disconnect from the terminal A of the three-way valve. Secondly, the sampling device is started to extract the sample gas to be measured (the flow rate is controlled within a stable range), so that the sample gas to be measured will enter the terminal C of the three-way valve from the terminal B of the three-way valve after being filtered by the dust filter, and will enter the infrared optical platform after the halogen is removed by the halogen removing device. The concentration value of CO₂ in the unoxidized sample gas to be measured will be detected in the infrared optical platform, to obtain the original concentration C1 of CO₂ before oxidation (after the measurement value is stable).

Further, the measuring the original concentration of CO₂ in the sample gas to be measured based on the infrared optical platform includes:
step a1, controlling an infrared light source to emit an infrared light with a preset wavelength to a gas chamber where the sample gas to be measured is located; and
step a2, obtaining an intensity after an infrared detector detects the infrared light, and calculating the original concentration of CO₂ in the sample gas to be measured based on the intensity.

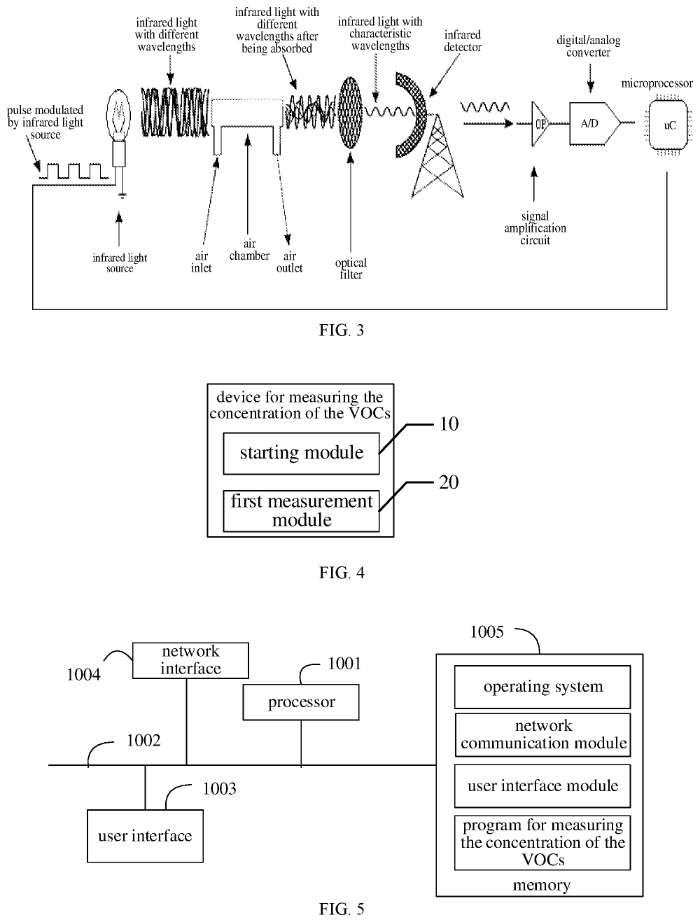

In this embodiment, as shown in FIG. 3, a narrow-band filter suitable for analyzing the absorption wavelength of the gas (such as the CO₂ in the present application) is installed in front of the infrared detector or the infrared light source, so that the signal changes of the infrared detector only reflect the concentration change of the sample gas to be measured. Taking the CO₂ analysis as an example, an infrared light with a preset wavelength (such as 1-20 um) is emitted from the infrared light source, and will pass through a narrow-band filter with a wavelength of 4.26μ m after being absorbed by a gas chamber with a certain length. Then an infrared detector will detect the intensity of the infrared light passing through the narrow-band filter with the wavelength of 4.26 μm, and the concentration of CO₂ in the sample gas to be measured can be calculated through the intensity.

In an embodiment, when the infrared light beam passes through the sample gas to be measured, the gas molecules in the sample gas to be measured will absorb the infrared light with a specific wavelength. As shown in FIG. 4, FIG. 4 is the infrared light absorption peak spectrum of CO₂. The absorption relationship obeys the Lambert-Beer law. The absorbance A corresponding to this absorption relationship can be obtained by the following formula:

$$A = lg ( \frac{{I}_{0}}{I} ) = lg ( \frac{1}{T} ) = kcd$$

I₀ is the intensity of the incident light, and I is the intensity of the transmission light after passing through the sample. c is the sample concentration of CO₂. d is the optical path, that is, the light transmission thickness (the length of the gas chamber) of the liquid tank for holding the solution. k is the proportional coefficient of light absorption. T is the transmittance, that is, the ratio of the intensity of the transmitted light to the intensity of the incident light.

It should be noted that when the concentration is the mole concentration, k is the mole absorption coefficient, which is related to the properties of the absorbing material and the wavelength \ of the incident light. It should be noted that when a beam of the parallel monochromatic light passes vertically through a uniform non-scattering light-absorbing material, the absorbance A is proportional to the concentration c of the light-absorbing material and the thickness d of the absorption layer.

The measuring the target conversion concentration of CO₂ includes:
step b, measuring a target conversion concentration of CO₂ based on the original concentration.

Further, the measuring a target conversion concentration of CO₂ based on the original concentration includes:
step b1, measuring a total concentration of CO₂ after conversion based on the infrared optical platform; and
step b2, calculating a difference between the total concentration after conversion and the original concentration to obtain the target conversion concentration.

In this embodiment, as shown in FIG. 2, the process of measuring the total concentration of the CO₂ after conversion in the sample gas to be measured is implemented through the following process. First, the terminal C of the three-way valve is controlled to connect with the terminal A of the three-way valve, and the terminal C of the three-way valve is controlled to disconnect from the terminal B of the three-way valve. Secondly, the sampling device is started, so that the sample gas to be measured will enter the gas-phase electrochemical reactor for performing gas-phase electrochemical oxidation after being filtered by the dust filter, and will enter the terminal C of the three-way valve from the terminal A of the three-way valve. Then the sample gas to be measured will enter the infrared optical platform after the halogen is removed by the halogen removing device. Finally, the concentration value of CO₂ in the sample gas to be measured will be detected in the infrared optical platform after passing through the gas-phase electrochemical reactor. The total concentration C₂ of the CO₂ after conversion after oxidation is measured after the value is stable.

The original concentration C₁ is subtracted from the total concentration C₂ after conversion to obtain the CO₂ concentration difference (Cdt) (the target conversion concentration), Cdt is used to calculate the carbon concentration of VOCs in the sample gas to be measured, and the carbon concentration is expressed as carbon concentration.

In an embodiment, VOCs can be expressed as the total hydrocarbons during the implementation of the specific standard and specification. Since the methane cannot be oxidized in the gas-phase electrochemical oxidation, the concentration value of the total VOCs expressed as the carbon concentration and calculated by using Cdt does not need to deduct the methane (CH4) concentration C_{CH4}, the concentration value of non-methane total hydrocarbons (NMHC) in the measured gas can be directly obtained, and the result is calculated in carbon.

It should be noted that, based on the infrared optical platform, the embodiment of measuring the total concentration of CO₂ after conversion is basically the same as the embodiment of measuring the original concentration of CO₂ in the sample gas to be measured based on the infrared optical platform, which will not be repeated here.

Further, before the starting the sampling device to inject the VOCs of the sample gas to be measured into the gas-phase electrochemical reactor for performing gas-phase electrochemical oxidation, and converting the VOCs into CO₂, the method for measuring the concentration further includes:
step c, calculating an influence value corresponding CO₂ in air.

Further, the influence value includes a zero point influence value and a range point influence value, and the calculating the influence value corresponding CO₂ in air includes:
step c1, calculating a zero point influence value corresponding to a zero gas with a first preset concentration and a range point influence value corresponding to a range standard gas with a second preset concentration respectively. In this embodiment, as shown in FIG. 2, the standard gas enters through the standard gas inlet, and the standard gas includes the zero gas and the range standard gas. In an embodiment, the influence of CO₂ on the measurement system is measured at the zero point and the range point. In an embodiment, after the measurement system runs stably, the test operation is performed. The test operation includes: first recording the indication value a₀ of the zero point and the indication value b₀ of the range point of the measurement system. Secondly, injecting the range standard gas with the specified concentration (the propane standard gas concentration is the full range value of 50%˜80%), and recording the indication values aᵢ and bᵢ of the measurement system. The range standard gas can be obtained through dilution. For the zero gas and the range standard gas with the same concentration, repeating the tests at a preset number of times (such as three or four times, and the like) according to the above test operation.

It should be noted that the first preset concentration includes 20.8% oxygen (the background gas is nitrogen, the indication value is a₀), 2000 ppm carbon dioxide mixed with 20.8% oxygen (the background gas is nitrogen, the indication value is a₁), and 5000 ppm carbon dioxide mixed with 20.8% oxygen (the background gas is nitrogen, and the indication value is a₂).

It should be noted that the second preset concentration includes propane mixed with 20.8% oxygen (the background gas is nitrogen, and the indication value is b₀), propane mixed with 2000 ppm carbon dioxide and mixed with 20.8% oxygen (the background gas is nitrogen, and the indication value is b₁), and propane mixed with 5000 ppm carbon dioxide mixed with 20.8% oxygen (the background gas is nitrogen, and the indication value is b₂).

It should be noted that the zero point influence value I_{z} is calculated by the following formula (1),

$${I}_{Z} = \frac{\overline{{a}_{i}} - \overline{{a}_{0}}}{R} \times 100 \% \qquad ( 1 )$$

a̅ᵢ̅ is the average value of the zero gas with the i-th concentration (for example, 2000 ppm carbon dioxide mixed with 20.8% oxygen) after a preset number of test operations. a₀ is the average value of the zero point (concentration of 20.8% oxygen) after a preset number of tests. R is the full-range value of the measurement system. i is the serial number of the recorded data (i=1˜2).

The range point influence value Iₛ is calculated by the following formula (2),

$${I}_{S} = \frac{\overline{{b}_{i}} - \overline{{b}_{0}}}{R} \times 100 \% \qquad ( 2 )$$

b̅ᵢ̅ is the average value of the range standard gas with the i-th concentration (such as propane mixed with 2000 ppm carbon dioxide and mixed with 20.8% oxygen) after a preset number of test operations. b̅₀̅ is the average value of the range point (the concentration is propane mixed with 20.8% oxygen) after a preset number of test operations. R is the full-range value of the measurement system. i is the serial number of the recorded data (i=1˜2).

Step d, if the influence value meets a preset requirement, performing the starting the sampling device to inject the VOCs of the sample gas to be measured into the gas-phase electrochemical reactor for performing gas-phase electrochemical oxidation, and converting the VOCs into CO₂.

In this embodiment, if the zero point influence value and the range point influence value both meet the requirements of ≤4% F.S, performing the starting the sampling device to inject the VOCs of the sample gas to be measured into the gas-phase electrochemical reactor for performing gas-phase electrochemical oxidation, and converting the VOCs into CO₂. If the zero point influence value and the range point influence value does not meet the requirements of ≤ 4% F.S, not performing the starting the sampling device to inject the VOCs of the sample gas to be measured into the gas-phase electrochemical reactor for performing gas-phase electrochemical oxidation, and converting the VOCs into CO₂.

Further, the VOCs are multi-component organic compounds, and the starting the sampling device to inject the VOCs of the sample gas to be measured into the gas-phase electrochemical reactor for performing gas-phase electrochemical oxidation, and converting the VOCs into CO₂ includes:
step e, starting the sampling device to separate the multi-component organic compound into a plurality of single-component organic compounds based on a chromatographic column, and injecting each of the plurality of single-component organic compounds into the gas-phase electrochemical reactor respectively to perform gas-phase electrochemical oxidation and convert the single-component organic compound into CO₂.

In this embodiment, by starting the sampling device, the sample gas to be measured containing VOCs is vaporized in the vaporization chamber and is brought into the chromatographic column through the inert gas (for example, the carrier gas, which also can be called the mobile phase). The chromatographic column is provided with a liquid phase or a solid state phase. Since different organic compounds in the VOCs of the sample gas to be measured have different boiling points, polarities or adsorption properties, each organic compound tends to form a distribution or adsorption equilibrium between the mobile phase and the state phase. Driven by the carrier gas, different components of the organic compound in VOCs will undergo repeated distribution or adsorption/desorption during movement, and will flow out of the chromatographic column successively, so that the analysis of different components of the organic compound can be achieved.

Different components of the organic compound will enter the gas-phase electrochemical reactor successively and will oxidize the generated CO₂.

Further, the method further includes:
step f, measuring a single-component conversion concentration of CO₂ corresponding to each of the plurality of single-component organic compounds respectively, to obtain a gas chromatogram.

In this embodiment, the amount (concentration) of CO₂ is measured through an infrared optical platform to obtain the single-component conversion concentration of CO₂ corresponding to each single-component organic compound. The amount of CO₂ is proportional to the amount or concentration of the measured component organic compound. The conversion concentration of each single component is displayed on the time axis to obtain a gas chromatogram.

The existing technology uses high-purity hydrogen to measure the concentration of VOCs, resulting in higher safety risks in the process of measuring the concentration of VOCs. Compared with that, in the present application, the sampling device is started to inject the VOCs in the sample gas to be measured to the gas-phase electrochemical reactor for performing gas-phase electrochemical oxidation and converting it into CO₂. The target conversion concentration of CO₂ is measured to obtain the carbon concentration of the VOCs in the sample gas to be measured. In the present application, the VOCs can be converted into CO₂ through a gas-phase electrochemical reactor. The gas-phase electrochemical reactor does not need to use the high-purity hydrogen when being used, so it does not involve the issue of safety management of flammable gases, thereby reducing the security risks in the process of measuring the concentration of VOCs.

In addition, the present application further provides a device for measuring the concentration of the VOCs. As shown in FIG. 4, the device for measuring the concentration of the VOCs includes:
a starting module 10 configured to start the sampling device to inject the VOCs in the sample gas to be measured into the gas-phase electrochemical reactor for performing gas-phase electrochemical oxidation and conversing the VOCs into CO₂; and
a first measurement module 20 configured to measure the target conversion concentration of CO₂ to obtain the carbon concentration of the VOCs in the sample gas to be measured.

In an embodiment, the device for measuring the concentration of the VOCs further includes:
a second measurement module configured to measure the original concentration of CO₂ in the sample gas to be measured based on the infrared optical platform.

The first measurement module 20 is further configured to measure a target conversion concentration of CO₂ based on the original concentration.

In an embodiment, the first measurement module 20 is further configured to measure a total concentration of CO₂ after conversion based on the infrared optical platform, and calculate a difference between the total concentration after conversion and the original concentration to obtain the target conversion concentration.

In an embodiment, the second measurement module is further configured to control an infrared light source to emit an infrared light with a preset wavelength to a gas chamber where the sample gas to be measured is located, obtain an intensity after an infrared detector detects the infrared light, and calculate the original concentration of CO₂ in the sample gas to be measured based on the intensity.

In an embodiment, the device for measuring the concentration of the VOCs further includes a calculation module configured to calculate an influence value corresponding CO₂ in air. If the influence value meets a preset requirement, the calculation module is configured to perform the starting the sampling device to inject the VOCs of the sample gas to be measured into the gas-phase electrochemical reactor for performing gas-phase electrochemical oxidation, and converting the VOCs into CO₂.

In an embodiment, the influence value includes a zero point influence value and a range point influence value, and the calculation module is further configured to calculate a zero point influence value corresponding to a zero gas with a first preset concentration and a range point influence value corresponding to a range standard gas with a second preset concentration respectively.

In an embodiment, the VOCs are multi-component organic compounds, and the starting module 10 is further configured to start the sampling device to separate the multi-component organic compound into a plurality of single-component organic compounds based on a chromatographic column, and inject each of the plurality of single-component organic compounds into the gas-phase electrochemical reactor respectively to perform gas-phase electrochemical oxidation and convert the single-component organic compound into CO₂.

In an embodiment, the device for measuring the concentration of the VOCs further includes a third measurement module configured to measure a single-component conversion concentration of CO₂ corresponding to each of the plurality of single-component organic compounds respectively, to obtain a gas chromatogram.

The specific implementation of the device for measuring the concentration of the VOCs of the present application is basically the same as the above-mentioned embodiments of the method for measuring the concentration of the VOCs, which will not be repeated here.

In addition, the present application further provides a device for measuring the concentration of the VOCs. As shown in FIG. 5, FIG. 5 is a schematic structural diagram of the hardware operating environment according to an embodiment of the present application.

It should be noted that FIG. 5 is a schematic structural diagram of the device for measuring the concentration of the VOCs under the hardware operating environment according to an embodiment of the present application.

As shown in FIG. 5, the device for measuring the concentration of the VOCs may include a processor 1001, such as a CPU, a memory 1005, a user interface 1003, a network interface 1004, and a communication bus 1002. The communication bus 1002 is configured to realize connection communication between these components. The user interface 1003 may include a display and an input unit such as a keyboard. The user interface 1003 may further include a standard wired interface and a wireless interface. The network interface 1004 may include a standard wired interface or a wireless interface (such as a WI-FI interface). The memory 1005 may be a high-speed RAM memory or a non-volatile memory, such as a disk memory. The memory 1005 may be a storage device independent of the aforementioned processor 1001.

In an embodiment, the device for measuring the concentration of the VOCs may further include a radio frequency (RF) circuit, a sensor, an audio circuit, a WiFi module, and the like.

Those skilled in the art can understand that the structure of the device for measuring the concentration of the VOCs shown in FIG. 5 does not constitute a limitation on the device for measuring the concentration of the VOCs, and may include more or less components than shown in the figure, or combine certain components, or a different component arrangement.

As shown in FIG. 5, the memory 1005 as the computer-readable storage medium, may include an operating system, a network communication module, a user interface module, and a program for measuring the concentration of the VOCs. The operating system is a program that manages and controls the hardware and software resources of the VOCs device for measuring the concentration, and supports the operation of the program for measuring the concentration of the VOCs and other software or programs.

In the device for measuring the concentration of the VOCs shown in FIG. 5, the user interface 1003 is mainly configured to connect to the terminal and communicate with the terminal. The network interface 1004 is mainly configured to connect to the background server and communicate with the background server. The processor 1001 may be configured to call the program for measuring the concentration of the VOCs stored in the memory 1005 and execute the method for measuring the concentration of the VOCs as described above.

The specific implementation of the device for measuring the concentration of the VOCs in the present application is basically the same as the above-mentioned embodiments of the method for measuring the concentration of the VOCs, which will not be repeated here.

In addition, embodiments of the present application further propose a computer-readable storage medium that stores a program for measuring the concentration of the VOCs. When the program for measuring the concentration of the VOCs is executed by a processor, the method for measuring the concentration of the VOCs as described above is implemented.

The specific embodiments of the computer-readable storage medium of the present application are basically the same as the above-mentioned embodiments of the method for measuring the concentration of the VOCs, which will not be repeated here.

It should be noted that, in the present application, the terms “comprising”, “comprises” or any other variations thereof are intended to cover a non-exclusive inclusion, such that a process, method, article or equipment that includes a series of elements not only includes those elements, but further includes other elements not expressly listed or inherent in the process, method, article or apparatus. Without further limitation, an element defined by the statement “comprises a . . . ” does not exclude the presence of additional identical elements in a process, method, article or apparatus that includes those elements.

The above serial numbers of the embodiments of the present application are only for description and do not represent the advantages or disadvantages of the embodiments.

Through the above description of the embodiments, those skilled in the art can clearly understand that the methods of the above embodiments can be implemented by means of software plus the necessary general hardware platform. Of course, it can further be implemented by hardware, but in many cases the former is better. implementation. Based on this, the essence of the technical solution of the present application or the part of the technical solution of the present application that contributes to the existing technology can be embodied in the form of a software product. The computer software product is stored in a storage medium (such as the ROM/RAM, the disk, the CD), including several instructions to cause a terminal equipment (which can be the mobile phone, the computer, the server, the equipment, or the network equipment, and the like) to execute the methods described in various embodiments of the present application.

The above-mentioned embodiments are only some embodiments of the present application, and are not intended to limit the scope of the present application. Any equivalent structure conversion or equivalent process conversion made with reference to the description and the accompanying drawings of the present application, directly or indirectly applied in other related technical fields, should all fall in the scope of the present application.

What is claimed is:

1. A method for measuring a concentration of volatile organic compounds (VOCs), comprising:
starting a sampling device to inject the VOCs of a sample gas to be measured into a gas-phase electrochemical reactor for performing gas-phase electrochemical oxidation, and converting the VOCs into carbon dioxide (CO₂); and
measuring a target conversion concentration of CO₂ to obtain a carbon concentration of the VOCs in the sample gas to be measured.

2. The method according to claim 1, wherein before the starting the sampling device to inject the VOCs of the sample gas to be measured into the gas-phase electrochemical reactor for performing gas-phase electrochemical oxidation, and converting the VOCs into CO₂, the method further comprises:
measuring an original concentration of CO₂ in the sample gas to be measured based on an infrared optical platform; and
the measuring the target conversion concentration of CO₂ comprises measuring a target conversion concentration of CO₂ based on the original concentration.

3. The method according to claim 2, wherein the measuring the target conversion concentration of CO₂ based on the original concentration comprises:
measuring a total concentration of CO₂ after conversion based on the infrared optical platform; and
calculating a difference between the total concentration after conversion and the original concentration to obtain the target conversion concentration.

4. The method according to claim 2, wherein the measuring the original concentration of CO₂ in the sample gas to be measured based on the infrared optical platform comprises:
controlling an infrared light source to emit an infrared light with a preset wavelength to a gas chamber where the sample gas to be measured is located; and
obtaining an intensity after an infrared detector detects the infrared light, and calculating the original concentration of CO₂ in the sample gas to be measured based on the intensity.

5. The method according to claim 1, wherein before the starting the sampling device to inject the VOCs of the sample gas to be measured into the gas-phase electrochemical reactor for performing gas-phase electrochemical oxidation, and converting the VOCs into CO₂, the method further comprises:
calculating an influence value corresponding CO₂ in air; and
in response to that the influence value meets a preset requirement, performing the starting the sampling device to inject the VOCs of the sample gas to be measured into the gas-phase electrochemical reactor for performing gas-phase electrochemical oxidation, and converting the VOCs into CO₂.

6. The method according to claim 5, wherein the influence value comprises a zero point influence value and a range point influence value, and the calculating the influence value corresponding CO₂ in air comprises:
calculating a zero point influence value corresponding to a zero gas with a first preset concentration and a range point influence value corresponding to a range standard gas with a second preset concentration respectively.

7. The method according to claim 1, wherein the VOCs are multi-component organic compounds, and the starting the sampling device to inject the VOCs of the sample gas to be measured into the gas-phase electrochemical reactor for performing gas-phase electrochemical oxidation, and converting the VOCs into CO₂ comprises:
starting the sampling device to separate the multi-component organic compounds into a plurality of single-component organic compounds based on a chromatographic column, and injecting each of the plurality of single-component organic compounds into the gas-phase electrochemical reactor respectively to perform gas-phase electrochemical oxidation and convert the single-component organic compound into CO₂.

8. The method according to claim 7, further comprising:
measuring a single-component conversion concentration of CO₂ corresponding to each of the plurality of single-component organic compounds respectively, to obtain a gas chromatogram.

9. A device for measuring a concentration of volatile organic compounds (VOCs), comprising a memory, a processor, and a program for measuring the concentration of the VOCs stored on the memory and executable on the processor, wherein when the program is executed by the processor, the method according to claim 1 is implemented.

10. A non-transitory readable storage medium, wherein a program for measuring the concentration of the VOCs is stored on the non-transitory readable storage medium, and when the program is executed by a processor, the method according to claim 1 is implemented.
